(19) United States
(12) Patent Application Publication
(10) Pub. No.: US 2016/0101267 A1
(43) Pub. Date: Apr. 14, 2016

(54) Anchoring Device and Methods for Use

(71) Applicant: Sanford Health, Sioux Falls, SD (US)

(72) Inventor: Patrick W. Kelly, Sioux Falls, SD (US)

(21) Appl. No.: 14/883,259

(22) Filed: Oct. 14, 2015

Related U.S. Application Data
(60) Provisional application No. 62/063,872, filed on Oct. 14, 2014.

(51) Int. Cl. A61M 25/09; A61F 2/95

(52) U.S. Cl. CPC A61M 25/09; A61F 2/95; A61M 2025/09125; A61M 2025/09066; A61M 2025/09083; A61F 2002/9511

(57) ABSTRACT

An anchoring guide wire and methods for use are provided, where the guide wire comprises a non-coiled segment, a first expandable segment, and a leading segment, wherein the first expandable segment is configured to transition between a constrained state and a deployed state.

### RELATED APPLICATIONS

This application is a non-provisional of and claims priority to U.S. Provisional Application No. 62/063,872 entitled “Anchoring Device and Methods for Use,” filed on Oct. 14, 2014, which is hereby incorporated by reference in its entirety.

### BACKGROUND OF THE INVENTION

The current state of the art is a steerable guide wire that may not achieve active fixation in a lumen in which it is deployed. In such a configuration, there may be no support for the wire in an environment such as a branch vessel arising from an aneurysm, and when a guide catheter is advanced over the wire, the wire may be pulled out of the target vessel. This may result in a longer surgery, thereby increasing the time that a patient is subjected to anesthesia, the operating room staff to more radiation, and the patient to more nephrotoxic intravenous contrast. In addition, losing wire access to a target vessel may result in the operator not being able to regain access resulting in the patient not having perfusion to that end organ as an outcome of the procedure.

### SUMMARY OF THE INVENTION

The present invention is directed to a device that improves anchoring of a steerable guide wire within a branched vessel, for example, by using an anchoring device to anchor a guide wire to the vasculature prior to advancement and deployment of an over-the-wire medical device. By improving the ease by which stent grafts may be placed in vessels (or other lumens, including, but not limited to ducts, orifices, the digestive tract, and/or any other tubular structure), the speed and success of complicated cases may increase while lowering complications. For example, anchoring the guide wire to a subject's vasculature may have a stabilizing effect for the tip of the catheter and afford greater stability and confidence for the operator of an implantable device to be deployed in vivo. Once the anchoring device is deployed, the anchor may hold the guide wire in place against the vasculature. One of the benefits of this design is that the anchoring device may allow blood to continue to flow through the anchor and to downstream vasculature. Once anchored, the guide wire may be used in a similar way to a through wire, which is a guide wire advanced into one access point such as the groin through and out of a second access point such as the arm. The advantage of a through wire can be secured on both ends preventing movement of the wire and providing support to the wire as the operator is working. A similar advantage can be achieved with the anchoring wire. This anchoring guide wire may be used for improving the delivery of over-the-wire therapeutic medical devices such as bare metal stents, covered stents, as well as any other over-the-wire device.

Thus, in a first aspect, the present invention provides a guide wire comprising a non-coiled segment, a first expandable segment and a leading segment, wherein the first expandable segment is configured to transition between a constrained state and a deployed state.

In a second aspect, the present invention also provides a method for deploying an anchoring device into a lumen, the method comprising: (a) introducing a guide wire according to the first aspect of the invention into a lumen via arterial access, wherein the guide wire is disposed in a catheter such that a first expandable segment of the guidewire is in a constrained state, and (b) transitioning the first expandable segment from the constrained state to a deployed state, thereby anchoring the guide wire in the lumen.

### BRIEF DESCRIPTION OF THE DRAWINGS

FIG. 1A is a side view of a guide wire in a constrained state, in accordance with one embodiment of the invention.

FIG. 1B is a side view of a guide wire in a deployed state, in accordance with one embodiment of the invention.

FIG. 1C is a side view of another guide wire in a deployed state, in accordance with one embodiment of the invention.

FIG. 1D is a side view of a guide wire disposed within a catheter, in accordance with one embodiment of the invention.

FIG. 1E is a detail cross-sectional side view of the guide wire introduced into a lumen, in accordance with one embodiment of the invention.

FIG. 2 is a flow chart depicting functions that can be carried out in accordance with example embodiments of the disclosed methods.

### DETAILED DESCRIPTION OF THE INVENTION

Exemplary devices and methods are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, with respect to measurements, “about” means +/−5%. Further, as used herein, “lumen” refers to a blood vessel or arterial configuration in which the anchoring device is deployed. The term “lumen” may further include artificial lumens used, for example, as teaching aids.

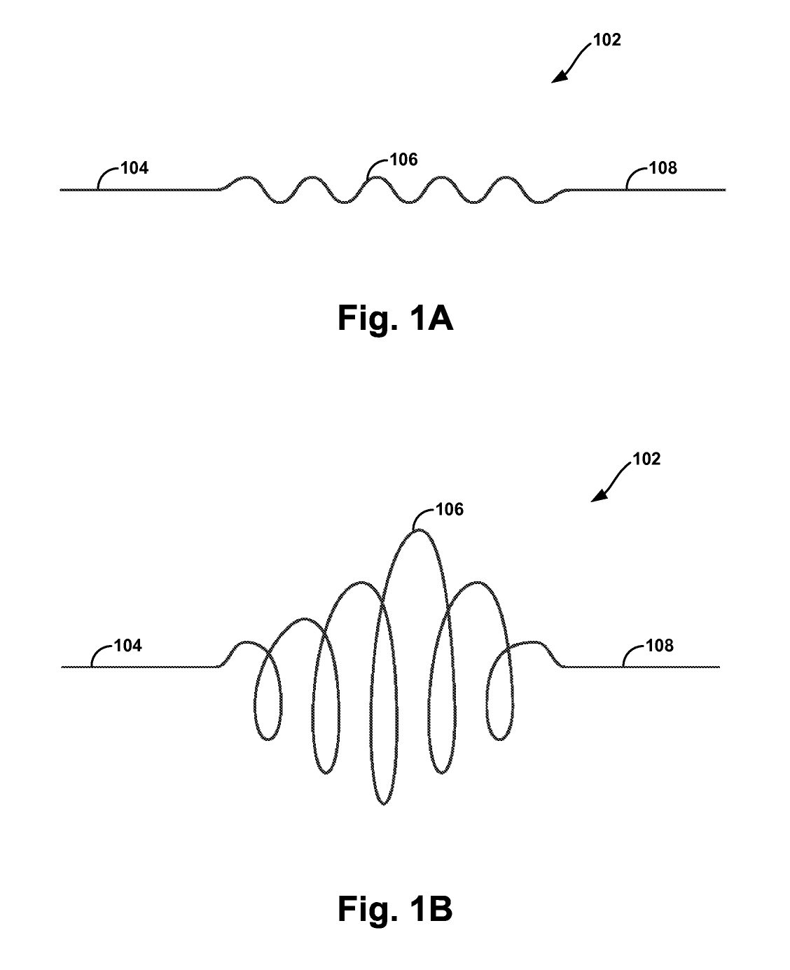

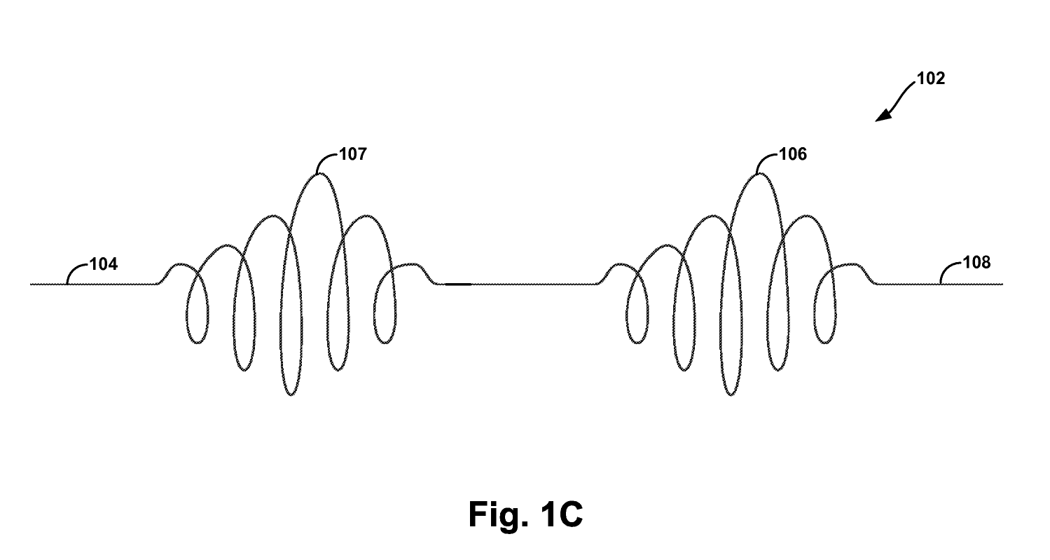

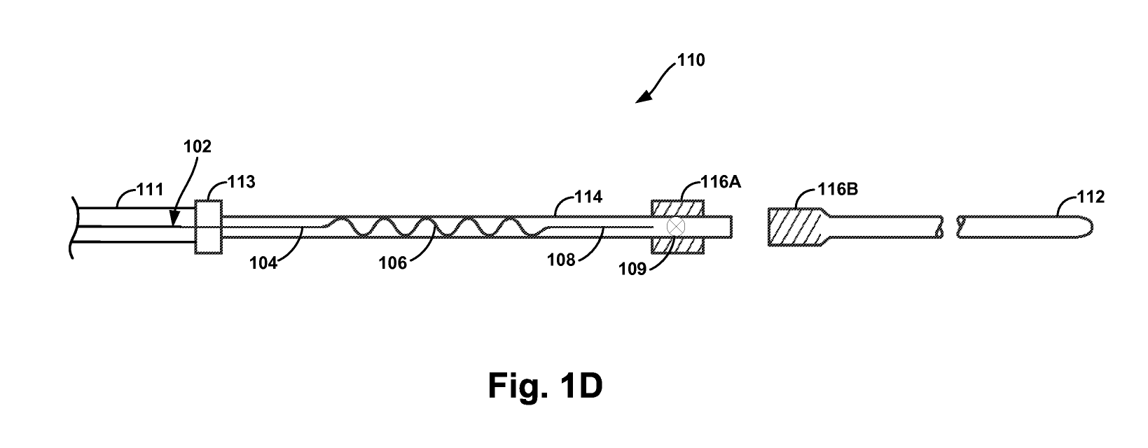

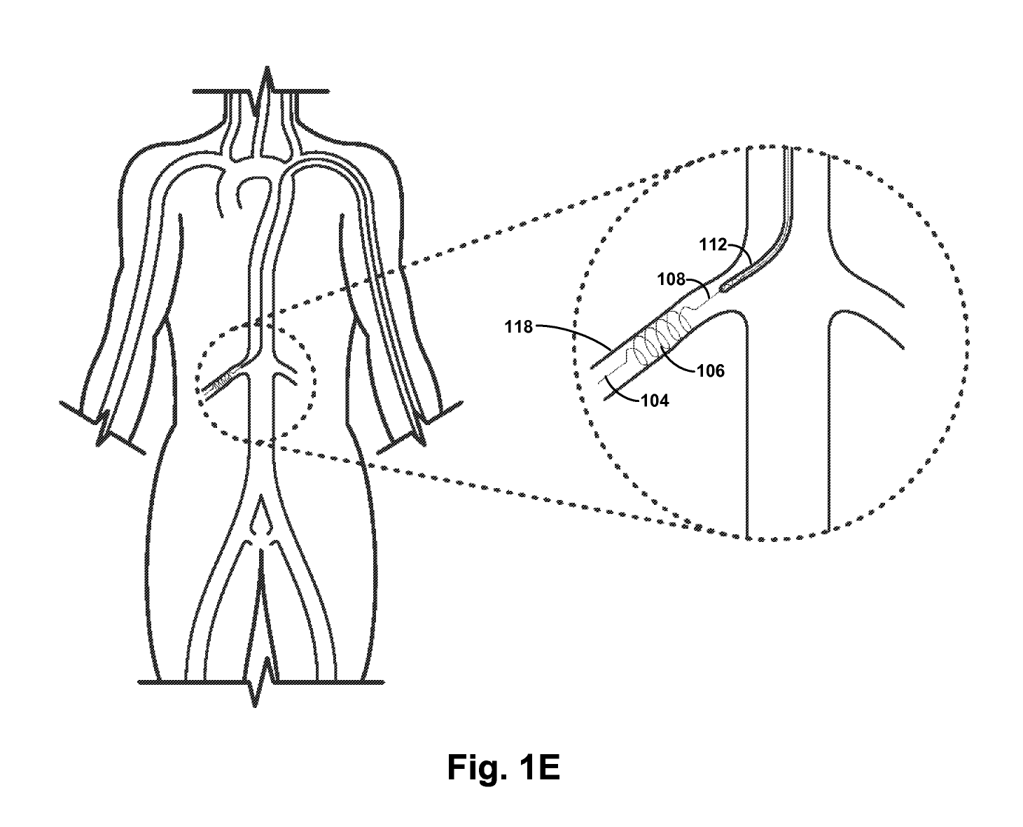

In a first aspect, FIG. 1A illustrates an example guide wire 102 in a constrained state. As shown in FIG. 1A, the guide wire 102 comprises a non-coiled segment 104, an expandable segment 106, and a leading segment 108. The non-coiled segment 104, the expandable segment 106, and the leading segment 108 may generally be co-axial and constructed of a single wire with a thickness in the range of about 0.01 in. to about 0.04 in. In some embodiments, the total length of the guide wire 102 may range from about 100 cm to about 500 cm, and preferably from about 100 cm to about 300 cm. The guide wire may be made of, for example, nitinol (nickel-titanium), titanium, titanium alloys, copper-aluminum-nickel alloys, various plastics, or any other suitable material capable of retaining shape memory.

The expandable segment 106 may be configured to exist in one of two different states: a constrained state (as shown in FIG. 1A) and a deployed state (as shown in FIG. 1B). As shown in FIGS. 1A and 1B, the expandable segment 106 has a smaller diameter in the constrained state than in the deployed state. In one embodiment, in the constrained state, the expandable segment 106 is substantially non-coiled and is disposed within a catheter 110, as illustrated in FIG. 1D. And in the deployed state, the expandable segment 106 may be coiled and configured to exert pressure against the walls of the vessel or lumen into which the device is introduced.

In some embodiments, when in the deployed state, the expandable segment 106 may exert a pressure against the vessel or lumen may range from about 0.25 ATM to about 3.0 ATM, and preferably from about 0.5 AMT to about 1.5 ATM. The pressure exerted by the expandable section 106 against the vessel or lumen should be substantially small such that it does not cause a dissection in the vessel or lumen but large enough that it provides an adequate amount of anchorage so that the wire does not slip out of the vessel or lumen. In the deployed state, the expandable segment 106 may have a length in the range of about 10 mm to about 100 mm, and an expanded diameter in the range of about 1 mm to about 80 mm. In one example, the expanded diameter of the expandable segment 106 in the deployed state may be approximately 20 percent larger than a diameter of the lumen in which the device is deployed. In some arrangements, different portions of the expandable segment 106 may exert different pressures. For instance, one portion of the expandable segment 106 may be a more tightly-wound coil and thereby exert more pressure against the vessel wall than other portions of the expandable segment 106. In another arrangement, coils of one portion of the expandable segment 106 may have a larger diameter and may therefore exert more pressure on the vessel wall than coils of another portion of the expandable segment 106. In another arrangement, one portion of the expandable segment 106 may be comprised of wires with a greater thickness that may exert more pressure against the vessel wall than other portions of the expandable segment 106. In still another arrangement, coils of one portion of the expandable segment 106 may be at a different pitch than coils of another portion of the expandable segment 106. Thus, one portion of the expandable segment 106 may exert a different pressure against the vessel wall than other portions. However, in other arrangements, each portion of the expandable segment 106 may exert substantially the same pressure as the other portions.

The leading segment 108 may extend beyond the expandable segment 106 in order to help guide the anchoring device 100 (while in the constrained state) through the lumen to a suitable deployment position. Additionally, the leading segment 108 may be shaped or constructed in such a way as to mitigate inadvertent injury to portions of the lumen with which the leading segment 108 comes into contact. For instance, a distal end of the leading segment 108 may be especially rounded or smoothed. Additionally or alternatively, the leading segment 108 may be constructed of a conformable material that is less stiff than other portions of the device, or less stiff than other, traditional in vivo materials. As such, the leading segment 108 may be considered “atraumatic.” The leading segment 108 has an additional benefit in that it is substantially straight, making loading the guide wire 102 into a catheter easier. The substantially straight leading segment 108 allows the operator loading the guide wire 102 into a catheter to get the guide wire 102 started in the catheter before trying to advance the expandable segment 106. The leading segment 108 also makes advancing the guide wire 102 through the catheter easier. If the terminal extent of the leading segment 108 were coiled, the coiled nature would push the tip into the inner surface of the catheter making advancement of the wire challenging. Instead, the substantially straight leading segment 108 enables the operator to more easily guide the guide wire 102 to the desired location. The leading segment 108 remains substantially straight even when the guidewire is in the deployed state, unless the wall of a vessel or lumen is acting upon the leading segment 108. As such, the leading segment need not be constructed from a shape memory material. In some examples, the leading segment 108 may extend beyond the expandable segment 106 at a length of about 2 mm to about 200 mm, and preferable a length of about 3 mm to about 50 mm. However, other examples are possible.

FIG. 1C is a side view of another anchoring device in a deployed state, in accordance with one embodiment of the invention. As shown in FIG. 1C, the guide wire 102 may further include a second expandable segment 107 positioned between the non-coiled segment 104 and the expandable segment 106. The second expandable segment 107 may be configured to transition between a constrained state and a deployed state similar to the expandable segment 106. In the constrained state, the second expandable segment 107 may be disposed within a catheter. In one embodiment, the second expandable segment 107 is arranged as a second coiled wire configured to exert pressure on the lumen in which the device is disposed. In some embodiments, the first expandable segment 106 and the second expandable segment 107 are arranged to exert the same pressure on a lumen; however, in other embodiments, the first expandable segment 106 is arranged to exert a different pressure on the lumen than the second expandable segment 107. Other examples are possible as well.

FIG. 1D illustrates the anchoring device 100 disposed within a catheter 110. In order to transition between the constrained state and the deployed state, the catheter 110 may be slid backward toward the non-coiled segment 104, thereby exposing the expandable segment 106 to the vasculature and allowing the expandable segment 106 to expand and coil thereby exerting an outward force on the walls of the vessel. In an alternate implementation, the device 100 may be pushed through the catheter 110 to advance and expose the expandable segment 106 in the same way. Likewise, in order to transition from the deployed state to the constrained state, the catheter 110 may be pushed back over the expandable segment 106; or, alternatively, the expandable segment 106 pulled back through the catheter 110. In yet another example, the expandable segment 106 is configured to transition between the constrained state and the deployed state by simultaneously sliding the expandable segment 106 out of the catheter 110 and sliding the catheter 110 over the expandable segment 106.

In some embodiments, the catheter 110 comprises a delivery catheter portion 112 and an interface catheter portion 114. The delivery catheter portion 112 may be advanced over a straight or steerable guide wire into the target vessel or lumen. As such, the delivery catheter is a substantially in vivo catheter. Once the straight or steerable guide wire is removed, with the delivery catheter 112 in place in the target vessel or lumen, the interface catheter 114 can be attached, and the guide wire 102 can be advanced through the delivery catheter 112 to the target vessel or lumen. As such, the interface catheter 114 may remain substantially ex vivo, and may be used to house the guide wire 102 prior to deployment. As such, the expandable segment 106 and the leading segment 108 may be disposed within the interface catheter portion 114 prior to use. The delivery catheter portion 112 may be shaped or constructed in such a way as to mitigate inadvertent injury to portions of the lumen with which the delivery catheter portion 112 comes into contact. For instance, the tip of the delivery catheter portion 112 may be especially rounded or smoothed. Additionally or alternatively, the delivery catheter portion 112 may be constructed of a conformable material that is less stiff than other portions of the device, or less stiff than other, traditional in vivo materials.

Before use, the non-coiled segment 104 may be positioned within a housing 111. The housing 111 may be a plastic material, for example. The housing 111 may be coupled to the interface catheter 114 via a locking/unlocking hub 113. In operation, the interface catheter 114 may be coupled to the delivery catheter 112 via complementary luer-lock connectors 116A, 116B, a threaded connector, or some other type of connector. Once the interface catheter 114 is coupled to the delivery catheter 112, the housing 111 may be decoupled from the locking/unlocking hub 113 and removed from surrounding the non-coiled segment 104 of the guide wire 102. Once the interface catheter 114 is connected to the delivery catheter 112 and the housing 111 is removed, a valve 109 on the leading edge of the interface catheter 114 can be opened allowing the operator to manipulate the non-coiled segment 104 of the guide wire 102 to thereby advance the guide wire 102 through the interface catheter 114 and into the delivery catheter 112, and eventually to the target vessel or lumen.

FIG. 1E illustrates a detail cross-sectional side view of the anchoring device introduced into a lumen 118. In accordance with one embodiment, in order to introduce the device 100 into the lumen, first a standard guide wire may be introduced into the lumen 118 via arterial access. Second, the delivery catheter 112 may be advanced into the lumen 118 over the standard guide wire until the delivery catheter 112 is in a desired location. At this point, the standard guide wire may be removed. Third, the interface catheter 114 may be coupled to an ex vivo end of the delivery catheter 112 (however, in some embodiments, the interface catheter 114 may already be coupled to the delivery catheter 112 at this point). Fourth, the expandable segment 106 and the leading segment 108 of the guide wire 102 are advanced through the interface catheter 114 and the delivery catheter 112, thereby deploying the expandable segment 106 in a desired location in the lumen 118, as shown in FIG. 1E. Fifth, the delivery catheter 112 is removed from the lumen 118, as shown in FIG. 1E leaving the guide wire 102 in position. Sixth, a therapeutic and/or implantable device, such as a stent along with its delivery catheter, is advanced over the non-coiled segment 104 of the guide wire 102 to a desired treatment location. It will be appreciated that other arrangements are possible as well, including some arrangements that involve more or fewer steps than those described above, or steps in a different order than those described above.

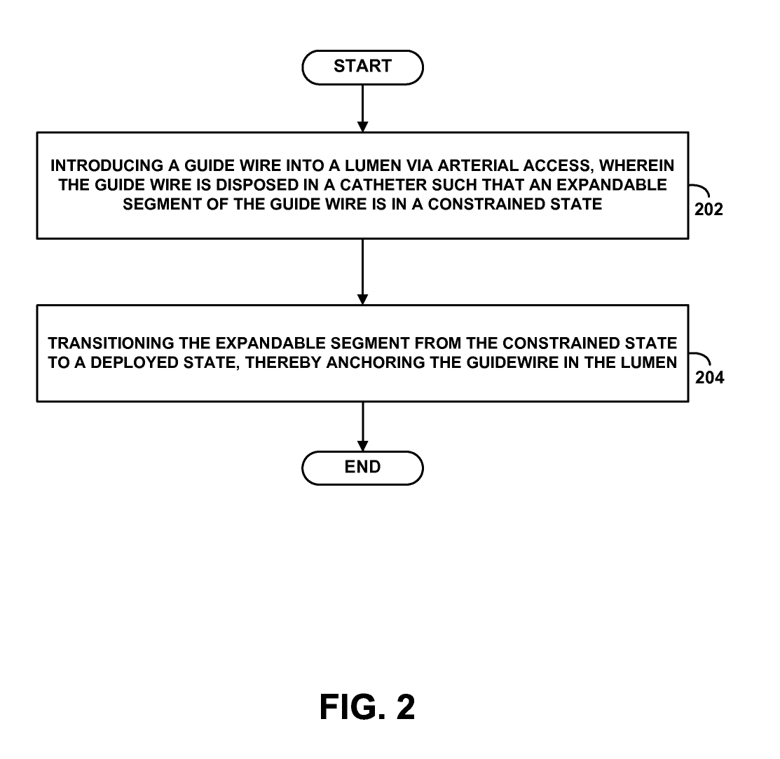

FIG. 2 is a simplified flow chart illustrating a method according to an exemplary embodiment. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 202, the method involves introducing the anchoring device as described above into a lumen via arterial access, wherein a guide wire of the anchoring device is disposed in a catheter such that an expandable segment is in a constrained state. The anchor device may be introduced into the lumen over a previously placed guide wire with the expandable segment in a constrained state in which the expandable segment is disposed within a catheter in a substantially non-coiled arrangement. In one example, introducing the device into the lumen comprises introducing a delivery catheter into a lumen via arterial access, the delivery catheter being coupled to an interface catheter having disposed therein a guide wire comprising a non-coiled segment, a first expandable segment and a leading segment. In such an example, the interface catheter may be coupled to the delivery catheter via a luer-lock connector, a threaded connector, or some other type of connector. Once the interface catheter is connected to the delivery catheter, a valve on the leading edge of the interface catheter can be opened allowing the anchoring wire to be advanced through the interface catheter and into the delivery catheter.

At block 204, the method involves transitioning the expandable segment from the constrained state to a deployed state thereby anchoring the guide wire in the lumen. As described above, transitioning the expandable segment forward from the constrained state to the deployed state may include sliding the expandable segment out of the catheter; or, alternatively or simultaneously, sliding the catheter back over the expandable segment. As the expandable segment becomes exposed in the deployed state, it may coil and expand due to shape memory, thereby exerting pressure on the lumen. The pressure exerted on the lumen in the expanded state where the expandable segment comes into contact with the vessel wall may range from about 0.25 ATM to about 3.0 ATM. As further described above, the expandable segment may exert a graduated pressure on the lumen such that a first portion of the expandable segment exerts a different pressure than a second portion of the expandable segment. In an alternate embodiment, each portion of the expandable segment may exert substantially the same pressure on the lumen.

The method may further include removing the catheter from the lumen while leaving the guide wire behind, and introducing a therapeutic and/or implantable device, such as a stent, over the non-coiled segment of the guide wire to a desired treatment location. It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the invention can be combined unless the context clearly dictates otherwise. The various aspects and embodiments disclosed herein are for purposes of illustration and are not intended to be limiting, with the true scope and spirit being indicated by the following claims.

1. A guide wire comprising:
a non-coiled segment, a first expandable segment, and a leading segment, wherein the first expandable segment is configured to transition between a constrained state and a deployed state.

2. The guide wire of claim 1, wherein the guide wire has a thickness in the range of 0.01 in. to 0.04 in.

3. The guide wire of claim 1, wherein the leading segment has a length in a range from about 2 mm to about 200 mm.

4. The guide wire of claim 1, wherein the guidewire has a length in a range from about 100 cm to about 500 cm.

5. The guide wire of claim 1, wherein the first expandable segment has a length in the deployed state in a range from about 10 mm to about 100 mm.

6. The guide wire of claim 1, wherein the first expandable segment has a diameter in the deployed state in a range from about 1 mm to about 80 mm.

7. The guide wire of claim 1, wherein the leading segment is substantially straight in both the constrained state and the deployed state.

8. The guide wire of claim 1, wherein the leading segment has a stiffness that is less than a stiffness of the non-coiled segment and the first expandable segment.

9. The guide wire of claim 1, wherein in the deployed state the first expandable segment is arranged as a coiled wire configured to exert pressure on a lumen.

10. The guide wire of claim 9, wherein in the deployed state, coils of the first expandable segment exert pressure on the lumen in a range from about 0.25 ATM to about 3.0 ATM.

11. The guide wire of claim 9, wherein in the deployed state coils of a first portion of the first expandable segment have a larger diameter than coils of a second portion of the first expandable segment such that the first portion of the first expandable segment is configured to exert a different pressure on the lumen than the second portion of the first expandable segment.

12. The guide wire of claim 9, wherein a first portion of the first expandable segment has a wire thickness that is greater than a second portion of the first expandable segment such that the first portion of the first expandable segment is configured to exert a different pressure on the lumen than the second portion of the first expandable segment.

13. The guide wire of claim 9, wherein in the deployed state coils of a first portion of the first expandable segment have a first pitch, and wherein in the deployed state coils of a second portion of the first expandable segment have a second pitch that is different than the first pitch such that the first portion of the first expandable segment is configured to exert a different pressure on the lumen than the second portion of the first expandable segment.

14. The guide wire of claim 1, wherein the guide wire further comprises a second expandable segment positioned between the non-coiled segment and the first expandable segment, wherein the second expandable segment is configured to transition between a constrained state and a deployed state.

15. The guide wire of claim 14, wherein, in the deployed state, the second expandable segment is arranged as a second coiled wire configured to exert pressure on the lumen.

16. The guide wire of claim 1, wherein the first expandable segment comprises a shape memory wire.

17. The guide wire of claim 16, wherein the shape memory wire comprises a material selected from the group consisting of including nitinol, titanium, titanium alloys, or copper-aluminum-nickel alloys.

18. The guide wire of claim 1, wherein the first expandable segment in a constrained state is disposed within a catheter.

19. The guide wire of claim 18, wherein the catheter comprises:
an interface catheter; and
a delivery catheter coupled to the interface catheter.

20. A method comprising:
introducing a guide wire according to claim 1 into a lumen via arterial access, wherein the guide wire is disposed in a catheter such that a first expandable segment is in a constrained state; and
transitioning the first expandable segment from the constrained state to a deployed state, thereby anchoring the guide wire in the lumen.

21. The method of claim 20, wherein, in the deployed state, the first expandable segment exerts pressure on the lumen.

22. The method of claim 20, wherein transitioning the first expandable segment from the constrained state to the deployed state comprises sliding the first expandable segment out of the catheter and expanding the first expandable segment due to shape memory.

23. The method of claim 20, further comprising:
transitioning the first expandable segment from the deployed state to the constrained state.

24. The method of claim 23, wherein transitioning the first expandable segment from the deployed state to the constrained state comprises sliding the catheter over the first expandable segment and returning the first expandable segment to a substantially non-coiled arrangement.

25. The method of claim 20, wherein introducing the guide wire according to claim 1 into a lumen via arterial access comprises:
introducing a delivery catheter into a lumen via arterial access, the delivery catheter being coupled to an interface catheter having disposed therein a guide wire comprising a non-coiled segment, a first expandable segment and a leading segment.

26. The method of claim 25, wherein transitioning the first expandable segment from the constrained state to a deployed state comprises at least one of:
sliding the first expandable segment through the interface catheter, through the delivery catheter, and out of the delivery catheter, and
sliding the interface catheter and the delivery catheter over the first expandable segment.

27. The method of claim 20, further comprising:
exerting pressure on the lumen via coils of the first expandable segment in the deployed state, the pressure being in a range from about 0.25 ATM to about 3.0 ATM.

28. The method of claim 20, further comprising:
exerting a graduated pressure on the lumen via coils of the first expandable segment in the deployed state such that a first portion of the first expandable segment exerts a different pressure than a second portion of the first expandable segment.

29. The method of claim 20, wherein the guide wire further comprises a second expandable segment positioned between the non-coiled segment and the first expandable segment, the second expandable segment being configured to transition between a constrained state and a deployed state, wherein in the deployed state the second expandable segment is arranged as a second coiled wire configured to exert pressure on the lumen, the method further comprising:
exerting a graduated pressure on the lumen via coils of the second expandable segment in the deployed state such that a first portion of the second expandable segment exerts a different pressure than a second portion of the second expandable segment.

30. The method of claim 20, further comprising:
removing the catheter from the lumen; and
introducing a therapeutic device over the non-coiled segment via the lumen.
